(19) United States
(12) Patent Application Publication
(10) Pub. No.: US 2022/0362821 A1
(43) Pub. Date: Nov. 17, 2022

(54) ORGANIC WASTE TREATMENT

(71) Applicant: Andion Global Inc., Vancouver (CA)

(72) Inventors: Daniele CHIODINI, Vancouver (CA); Mauro CRIPPA, Missaglia (IT); Stefano STUCCHI, Paladina (IT)

(21) Appl. No.: 17/773,955

(22) PCT Filed: Nov. 6, 2019
(86) PCT No.: PCT/CA2019/051580

§ 371 Date: May 3, 2022

(51) Int. Cl. B09B 3/32; B01D 29/35; B01D 29/90; B01D 21/24; B01D 36/04; C12M 1/00; C12N 1/20

(52) U.S. Cl. CPC B09B 3/32; B01D 29/35; B01D 29/906; B01D 21/2461; B01D 36/04; C12M 45/04; C12M 45/03; C12M 23/40; C12N 1/20; B09B 2101/25

(57) ABSTRACT

An apparatus (2) and a method for pretreatment of organic waste from the Organic Fraction of Municipal Solid Waste and/or from food industries, upstream of an anaerobic digester. The apparatus comprises:—a squeezing-diluting-unpacking module (4) configured to receive an input waste stream (3) of packaged waste and a dilution fluid (10), to lacerate the packaging, and to output a coarser non-digestible stream (23) and a comparatively dirty and comparatively liquid slurry (26),—at least one settling module (30, 30a) configured to allow inerts in the comparatively dirty and comparatively liquid slurry (26) to sediment and to output a finer inerts stream (40) and a comparatively refined slurry (42),—a thickening/dewatering module (60) configured to separate and output a liquid stream (69) and a comparatively dewatered slurry (72).

### FIELD

The invention relates to the treatment of organic waste. The invention may be applied to treat the so-called Organic Fraction of Municipal Solid Waste (OFMSW) and industrial organic waste (e.g. production waste from food industries), but not including sewage sludge, agricultural biomasses, animal manure, civil waste water; this will be referred to in short as “organic waste” herein.

### BACKGROUND

Waste management presents significant problems. Increasing waste recycling and its exploitation is a world-wide goal; some objectives are to reduce the amount of waste to be disposed of in landfills, to increase the share of recycled material, and to achieve fully separate collection of organic waste.

Treatment and recycling of the various fractions constituting municipal waste (e.g. organic fraction, paper, metal, plastics, dry waste, etc.) contribute in a decisive way to the attainment of the above goal. In particular, the recycling of OFMSW, together with industrial organic waste, plays a fundamental role for several reasons, including the following ones:
a reduction in the amount of fermentable organic substances sent to landfills is particularly welcome and even requested by some regulations,
organic waste can have high energy potential. For example, organic waste may be treated at suitable plants to produce electricity and/or bio-methane through an anaerobic digestion process,
the specific kind of organic waste of concern herein is characterized by a high humidity and therefore a high specific weight, thus playing a major role in attaining yearly objectives referred to quantities/weights.

OFMSW is collected separately from other waste fractions in most Municipalities, and typically arrives at treatment plants in (supposedly biodegradable) plastic bags that need to be separated before the OFMSW is digested by microorganisms; in the case of industrial organic waste, there is a need for separation of packaging such as for example cardboard, plastic bags, Tetra pak® containers, plastic bottles, glass, jars and cans.

OFMSW, as well as industrial organic waste, includes e.g. fruit, vegetables, eggs, food leftovers, meat scraps etc. However, OFMSW usually also includes some undesired non-organic materials (e.g. smaller plastic, Tetra Pak®, paperboard and similar packaging, clothes and other fabrics, cutlery, glass, stones and gravel, etc.) due to incorrect separate collection. Furthermore, the kind of organic waste here of concern (OFMSW & industrial organic waste) usually includes hardly degradable organic materials such as bones, fruit shells, fruit seeds etc., that should be removed before digestion.

In the present description and in the following claims, “inerts” means those components that cannot be digested by microorganisms in the digester. It is noted that sewage, agricultural and animal waste typically does not contain any significant quantity of inerts.

U.S. Pat. No. 4,040,953 discloses a process for treating a liquid slurry of organic material, notably sewage and manure, to produce a gaseous product comprising a major amount of methane gas and a minor amount of carbon dioxide gas. The slurry is pulped in a wet pulping digester and mixed with recycled water and fresh water into a slurry with a minimum of 4% solids by weight. The slurry is passed from the wet pulping digester to a liquid cyclone for removal of grit and solids and then through a strainer to dewater the slurry to a minimum content of about 10%. The present Applicants believe that the wet pulper would not be suitable for dealing with packaged organic waste because plastic bags and other containers would accumulate in the wet pulper. Even if such packages managed to exit the pulper they would quickly clog the strainer, thus compromising the entire process.

AU2017200716A1 discloses a system and method for processing mixed waste. The described method comprising: comminuting a feedstock of mixed waste; dry separating the comminuted waste into an organic fraction and a non-organic fraction; adding water to the organic fraction to create a slurry; wet separating the organic fraction in the slurry into a more refined organic fraction and a residual non-organic fraction using relative densities of the more refined organic fraction and the residual non-organic fraction; and dewatering slurry containing the more refined organic fraction. Removal of plastic bags and other containers mainly takes place in a sorting substation, separately and upstream from the treatment of the organic waste.

Furthermore, the document is not specifically concerned with the dilution and dewatering steps.

WO2015/056073A1 discloses a device for the treatment of OFMSW, comprising a first module for forming a heterogeneous mixture of OFMSW in a longitudinal vat with auger for conveying the supplied OFMSW to a fixed cylindrical filter having open ends and encasing a second auger. The cylindrical filter is housed with an interspace in a vertical casing provided with a plurality of nozzles for directing jets of a liquid, for example water, for diluting the OFMSW onto the fixed cylindrical filter. A tubular fitting is provided in the casing for introducing the supplied OFMSW into the vertical filter through an opening provided at the lower end of the cylindrical filter. An opening for discharging the coarsest inerts is provided at the upper end of the filter. The device includes a second module for homogenizing the heterogeneous mixture of OFMSW, above which the first module is mounted. The second module has a first chamber arranged below the cylindrical filter and in which there directly falls—by gravity—the heterogeneous mixture of OFMSW coming through the lower end of said cylindrical filter, in which first chamber there is provided a mixture and removal auger and an auger for the transfer and removal of the residue inerts. The first chamber is followed by a second chamber having a greater longitudinal extension, in which there is extended said auger for the transfer and removal of the first chamber for conveying and removing the finest inerts. There is also provided a third chamber for further mixing the matrix in the mixture taken in a homogenization stage substantially liquid and dischargeable through the discharge end, wherein between the second chamber and the third chamber there is provided an overflow opening. According to this document, it is advantageous that the output of the second module can be directly pumped into the digester thanks to its liquid state.

There remains a need for better methods and apparatus for treating organic waste, especially waste having the characteristics of OFMSW and industrial organic waste.

### SUMMARY

A technical problem at the basis of the invention is to provide an efficient and cost-effective pretreatment—upstream of a digester- of organic waste of the kind discussed above, as separately collected. A more specific aim of the invention is that of rendering the pretreatment less sensitive to the variability of dry matter content of the waste.

In an aspect the invention relates to an apparatus for pretreatment of organic waste from the Organic Fraction of Municipal Solid Waste and/or from food industries, upstream of an anaerobic digester. The apparatus includes:
a squeezing-diluting-unpacking module configured to receive an input waste stream of packaged waste and a dilution fluid, to lacerate the packaging, and to output a coarser non-digestible stream and a comparatively dirty and comparatively liquid slurry, the coarser non-digestible stream may, for example, include plastic packages, Tetrapack™ packages, non-squeezable packing and undesired materials like textile, metals wood, etc.; The sizes of the pieces of material in the coarser non-digestible stream may, for example, be in the range of about 2 cm to 30 cm. The comparatively liquid slurry may carry some inerts. The inerts in the slurry are typically smaller than about 20 mm.
at least one settling module configured to allow inerts in the comparatively dirty and comparatively liquid slurry to sediment and to output a finer inerts stream and a comparatively refined slurry,
a thickening/dewatering module configured to separate and output a liquid stream and a comparatively dewatered slurry.

In an aspect the invention relates to a plant for pretreatment of organic waste from the Organic Fraction of Municipal Solid Waste and/or from food industries comprising an apparatus as discussed above, and an anaerobic digester downstream thereof.

In an aspect the invention relates to a method for pretreatment of organic waste from the Organic Fraction of Municipal Solid Waste and/or from food industries, upstream of an anaerobic digester, comprising:
a) receiving a packaged waste stream,
b) squeezing and diluting the packaged waste stream while unpacking the waste to output a coarser non-digestible stream and a comparatively dirty and comparatively liquid slurry,
c) letting comparatively finer inerts in the comparatively liquid slurry sediment to output a finer inerts stream and a comparatively refined slurry,
d) thickening/dewatering the comparatively refined slurry to output a liquid stream and a comparatively dewatered slurry.

Advantageous embodiments are disclosed in the dependent claims.

Further aspects of the invention and features of example embodiments are described below and/or illustrated by the accompanying drawings.

### BRIEF DESCRIPTION OF THE DRAWING

Further features and advantages of the present invention will be more clearly apparent from the following detailed disclosure of some embodiments thereof, made with reference to the attached drawings, which are not necessarily in scale, and wherein:

FIG. 1 is a block diagram of an embodiment of an anaerobic treatment plant for organic waste of the kind here of concern, including an apparatus according to the invention,

FIG. 2 is a diagrammatic view of an embodiment of a first module of the apparatus according to the invention,

FIG. 3 is a diagrammatic view of an embodiment of a second module of the apparatus according to the invention,

FIG. 4 is a diagrammatic view of an embodiment of a third module of the apparatus according to the invention,

FIGS. 5 and 6 are diagrammatic views of two embodiments of another module of the apparatus and plant according to the invention,

FIG. 7 is a diagrammatic view of an embodiment of the apparatus according to the invention, and

FIG. 8 is a flow chart illustrating a method according to an example embodiment of the invention.

### DETAILED DESCRIPTION

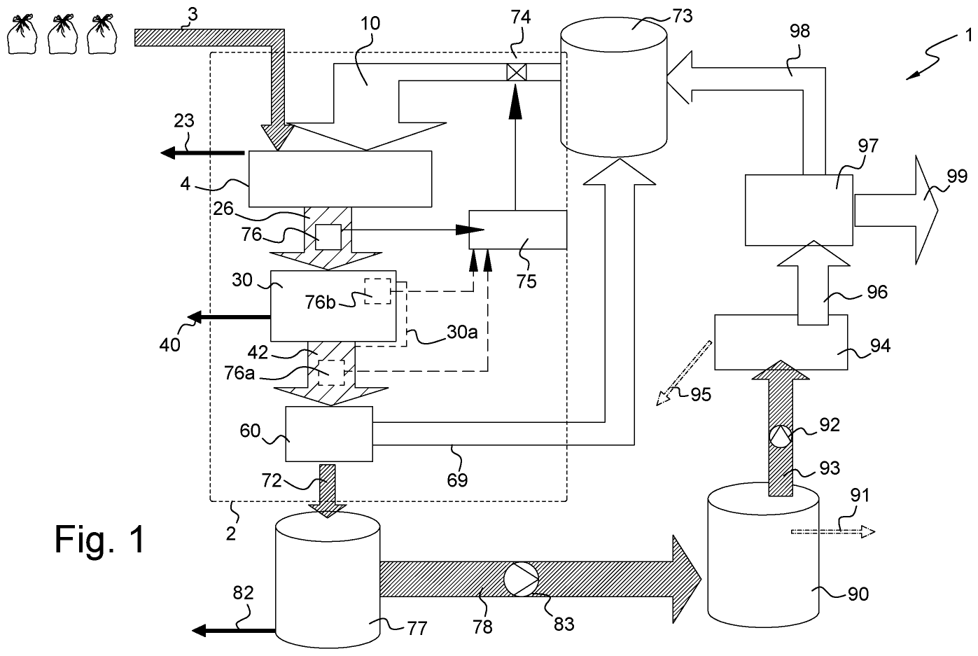

In FIG. 1 there is shown, schematically and only by way of an example, an embodiment of an anaerobic treatment plant 1 for wet organic waste, including an apparatus 2 according to an embodiment of the invention. Solely for the sake of clarity, in the arrows of the block diagram:
a comparatively dry stream of organic waste is indicated with densely hatched arrows,
a comparatively diluted (wet) stream of organic waste is indicated with less densely hatched arrows,
a stream of a dilution fluid/water is indicated with empty arrows,
a stream of inerts is indicated with solid line arrows,
a stream of valuable matter is indicated with dashed line arrows.

Wet organic waste, sometimes also referred to as “wet fraction” hereinbelow, be it from food industries and/or from solid municipal waste (Organic Fraction of Municipal Solid Waste, OFMSW), should in principle contain just digestible organic matter, such as fruit, vegetables, meat, fish, bread, eggs, cheese, coffee grounds, etc. However, wet organic waste arrives at plant 1, that includes an apparatus 2, e.g. by truck-in plastic bags and/or other containers such as paper, Tetra Pak®, cardboard containers, glass, jars, cans etc.

There might be an optional pretreatment (not shown) such as manual or mechanical separation of contents other than organic matter, before insertion, as a packaged waste stream 3, into the first component of the plant 1, namely first or squeezing-diluting-unpacking module 4. Such optional pretreatment separation may be automatic, manual or a combination thereof.

The packaged waste stream 3 usually has a dry matter content of about 15% to about 40% and typically has a dry matter content in the range of about 25-30%. Within such range, the dry matter content is highly variable since it depends on the source of waste stream 3 and on what people discard every day.

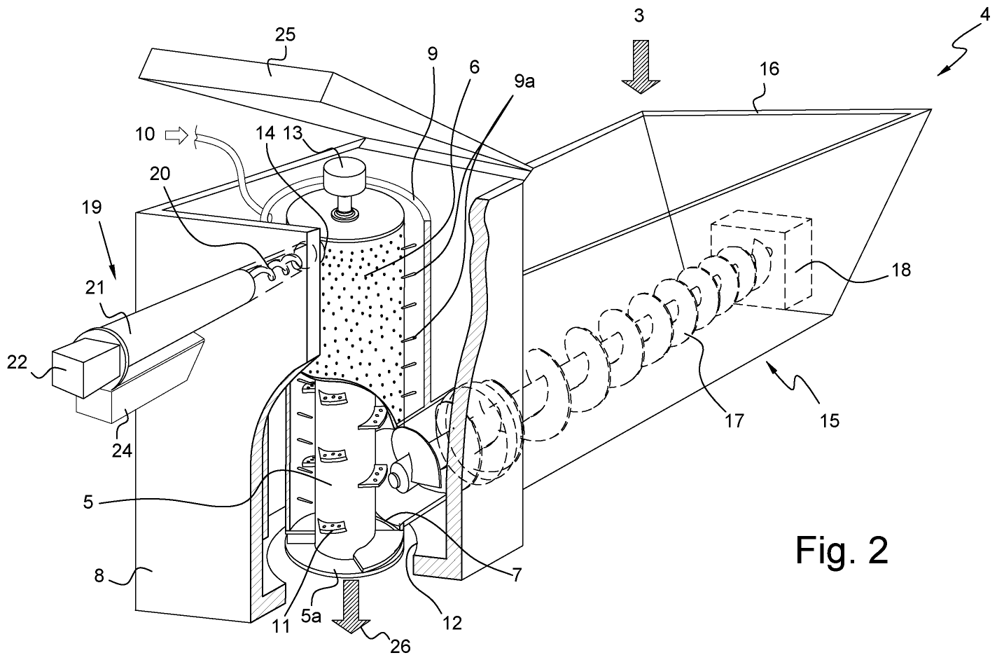

According to an embodiment thereof shown in FIG. 2, first or squeezing-diluting-unpacking module 4 includes an upwardly conveying worm screw 5 rotating within an open-bottom cylindrical filter 6 (only a portion of which is shown in order not to obscure the underlying components). The wet fraction of packaged waste stream 3 enters filter 6 at inlet 7 near its bottom. Filter 6 is also enclosed by a nozzled manifold 9 for inletting a dilution fluid 10 (FIG. 1), which is sprayed inwards of the filter 6, wherein it mixes with the wet fraction. Only a few nozzles 9a are visible in FIG. 9 for the purpose of illustration. Nozzles 9a are preferably evenly distributed around filter 6.

Nozzles 9a may, for example, be supplied with fluid at a working pressure in the range of about 1 to 3 bar (e.g. about 2 bar). An example embodiment includes 5 to 20 nozzles (e.g. 8 nozzles). The number, size(s) and flow rate(s) of the nozzles may be selected to allow the total flow of liquid provided by the nozzles to be adjusted within a desired range of flow rates. Nozzles 9a may, for example, have sizes in the range of about 2½ to 5 cm (about 1 to 2 inches), e.g. 3.75 cm (1½ inches). In an example implementation, each nozzle may be operated to provide a flow in the range of from 100 l/min to 200 l/min. Nozzles 9a may have various spray profiles. In some implementations nozzles 9a provide conical spray (e.g. “full cone” spray pattern). The width of the pattern may be varied. In some implementations the nozzle opening angle is about 60°.

In some implementations, each nozzle has a corresponding sectioning valve. The sectioning valves may be applied to turn individual nozzles 9a on or off and/or to adjust the flow rate of individual nozzles 9a.

The thread of worm screw 5 is periodically interrupted so that a plurality of blades 11 is formed, each having a cutting edge. A disc 5a sized to rotate within filter 6 is carried by worm screw 5. Disc 5a is located close to the bottom of filter 6, while forming a gap with the boundary of the opening 12 at the bottom of a case 8 of first module or squeezing-diluting-unpacking module 4.

While worm screw 5 rotates driven by a motor 13, the wet fraction is ground thereby, mixed with sprayed dilution fluid 10 (dilution function), centrifuged and filtered through filter 6 (squeezing function). Squeezed material is driven through filter 6 by centrifugal force. A casing around filter 6 collects the material that passes out through filter 6. This material then falls by gravity toward and through opening 12 at the bottom of case 8. Worm screw 5 may, for example operate at a speed in the range of about 550 rpm to about 2100 rpm (e.g. 850 rpm).

Filter 6 has openings of a size that may be selected according to the waste to be treated. The openings typically have sizes in the range of 10 mm to 30 mm. Openings having diameters of about 25 mm are good for many cases. Filter 6 may, for example, comprise a metal sheet perforated with suitably sized holes or a suitable mesh. In some cases the holes are rounded or circular.

Meanwhile, blades 11 lacerate and break any plastics, Tetra Pak®, paperboard and similar containers and packaging. Furthermore, blades 11 carry the pieces of such materials, as well as any non-grinded organic pieces, having a size larger than the openings in filter 6, upward toward a discharge outlet 14 near the top of filter 6 (unpacking function).

Dilution fluid 10 also serves to wash those containers and bags, so as to efficiently separate them from the wet fraction to be treated. The dilution fluid 10 can advantageously be totally recirculated dirty water as discussed below, and is sometimes simply called “water” below.

More in detail, in the embodiment shown, first module 4 preferably further comprises a feeder 15 for feeding waste stream 3 into inlet 7. Feeder 15 may comprise, for example, a basin 16 with tapered walls and with a horizontal axis worm screw 17 housed at its bottom. Waste from packaged waste stream 3 is dropped in batches into basin 16, and can then be continuously fed at a desired rate into inlet 7 while worm screw 17 rotates driven by a motor 18.

Furthermore, first module 4 preferably further comprises an extraction screw conveyor 19, comprising a worm screw or auger 20 inside a hollow pipe 21, driven to rotate by a motor 22, that conveys away the stream of plastic bags, packages and non-squeezable material, named coarser non-digestible stream 23 herein and in the following claims (FIG. 1), e.g. toward a container 24 for later disposal as shown.

A liftable cover 25 may selectively allow access from above to the cylindrical filter 6 for unclogging and maintenance reasons.

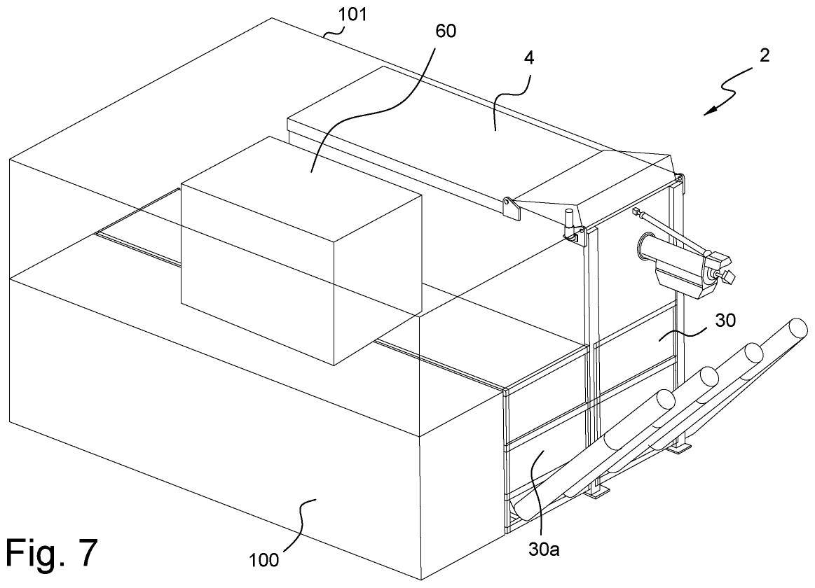

Turning back to FIG. 1, the centrifuged, diluted wet fraction that exits through opening 12 of cylindrical case 8 forms a slurry 26 that enters a second or settling module 30. Preferably, second or settling module 30 is placed directly below first module 4, e.g. as illustrated in FIG. 7, but alternatively any transportation arrangement, and possibly a pump (not shown) between the two modules may be provided.

Slurry 26 is a quite liquid heterogeneous mixture that still includes some finer inerts that have passed through filter 6. These remaining inerts are of a size less than the filter holes (e.g. less than 10-30 mm). The inerts still present are usually rather dense; e.g. they typically comprise glass, shellfish, and fruit seeds and similar.

The dry matter content of slurry 26 is typically about 5%. Maintaining the dry matter content of slurry 26 in the range of about 4% to about 6% provides a good balance of efficiency and cost. Allowing the dry matter content of slurry 26 to exceed about 6% tends to reduce the efficiency with which inerts can be removed by downstream processes (discussed below). Operating with slurry 26 at a dry matter content of less than 4% can help to improve the efficiency with which inerts can be separated and tends to increase the volume of slurry 26 for a given input waste stream 3. This adds expense because downstream equipment must be larger in size to accommodate the increased flow of slurry 26.

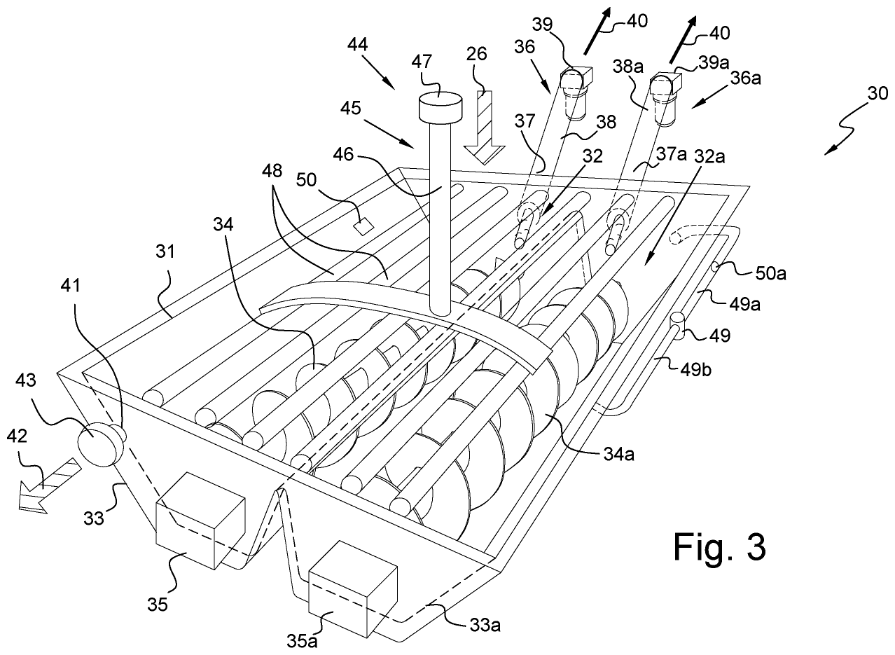

According to an embodiment thereof shown in FIG. 3, second or settling module 30 includes an elongate basin 31, which bottom portion is preferably divided into two chambers 32, 32a with tapered walls 33, 33a. Two parallel, worm screws or augers 34, 34a extend on the bottom of a respective chamber 32, 32a. Worm screws 34, 34a are driven to rotate slowly by respective motors 35, 35a (for example, screws 34, 34a may be driven to rotate at a speed in the range of about 5 rpm to about 50 rpm (e.g. 20 rpm). Screws 34, 34a operate to collect the finer inerts (of a size smaller than the holes of filter 6) that sediment from slurry 26.

Second or settling module 30 further comprises two extraction inclined screw conveyors 36, 36a, each comprising a worm screw or auger 37, 37a inside a hollow pipe 38, 38a driven to rotate by a motor 39, 39a. Conveyors 36, 36a convey away the finer inerts stream 40 (FIG. 1) for later disposal. Inerts stream 40 may comprise materials such as sand, grit, glass, bones, fruit seeds and shells etc.

It is noted that the horizontal worm screws 34, 34a push the material and directly feed the inclined screw conveyors 36, 36a, thus advantageously avoiding clogging, as conversely occurs frequently in conventional sand removal units.

More than two chambers 32, 32a may be formed, each additional chamber being provided with the components described above for chambers 32, 32a. Alternatively, settling module 30 may include only a single chamber 32 with its associated components.

When second module 30 is placed directly below first module 4 (such as in FIG. 7), basin 31 has preferably an open ceiling and is filled by the incoming slurry 26 that falls by gravity from first module 4; otherwise basin 31 may include an inlet for receiving the incoming slurry 26.

An outlet 41 for a slurry 42 (FIG. 1) to be fed to later described third module 60 (FIG. 1) is provided slightly below the level of slurry contained, operatively, in basin 31. A pump 43, preferably a centrifugal pump, may be provided at outlet 41.

Second or settling module 30 may further comprise an air mixing system 44 to avoid the stratification of material in basin 31 and formation of a crust. Air mixing system 44 comprises a manifold 45 whose bigger pipe 46 is connected to a blower 47, and whose smaller pipes 48 have perforations (not visible) therealong, to allow formation of large air bubbles. While six smaller pipes 48 are shown by way of an example, their number may be properly selected.

Second or settling module 30 may further comprise a recirculation pump 49 and/or a grinder (not shown). Recirculation pump 49 is preferably a centrifugal pump that intakes slurry 26 that has just entered second or settling module 30, e.g. from an intake pipe 49a entering basin 31 just below the level of slurry contained, operatively, in basin 31, and pumps slurry 26 along a recirculation pipe 49b (or more than one) running externally along basin 31, and again into basin 31 essentially at the same height, but in another position. This arrangement allows the slurry to better amalgamate.

Preferably, second module 30 further comprises a dry matter content sensor 50 dipped in basin 31, or a dry matter content sensor 50a arranged in recirculation pipe 49a, better disclosed below.

Optionally, an additional settling module 30a (FIG. 1) may be present, in series with second or settling module 30. In that case, the slurry 42 pumped out of settling module 30 is input to additional settling module 30a, while the slurry pumped out of additional settling module 30a forms the slurry 42 input to the third module 60. Alternatively, transfer of the slurry from settling module 30 to additional settling module 30a takes place through an overflow opening in which case it is not necessary to provide pump 43 in settling module 30.

According to an alternative embodiment (not shown), second or settling module 30 might include a first chamber where the slurry 26 output from first module 3 enters and stays for a short time, and where a relatively fast worm screw removes residual large inerts; a second elongate chamber with a relatively slow worm screw, that allows sedimentation and discharge of smaller, finer, or heavier inerts; and a third chamber where final mixing takes (passively) place, and from which the slurry is pumped out towards third module 60. The relatively slow worm screw may further extend into the first chamber for also performing the function of conveying the slurry to the second elongate chamber. From the second to the third chamber, transfer may occur through an overflow opening. Air or water may be introduced into the second chamber in order to avoid surface aggregations of inerts.

Turning back to FIG. 1, between the input to plant 1 and the output of settling module 30, about 70% to 90% of the inerts are preferably removed. That is, slurry 42 output from settling module 30 is much more refined than stream of packaged waste 3. Slurry 42 is referred to as being comparatively refined, and slurry 26 is referred to as being comparatively dirty herein and in the attached claims.

Slurry 42 output from settling module 30 and input to third module 60 is essentially comprised of organic matter without inerts, and is essentially liquid. The dry matter content of slurry 42 is similar to the dry matter content of slurry 26 since removal of inerts does not typically have a significant effect on the dry matter content (e.g. in the range of about 4% to about 6%). It is preferable to maintain the dry matter content of slurry 42 at about 5%. In order to improve efficiency and/or reduce costs of the downstream digestion of the slurry, such slurry 42 is fed to third or thickener/dewatering module 60.

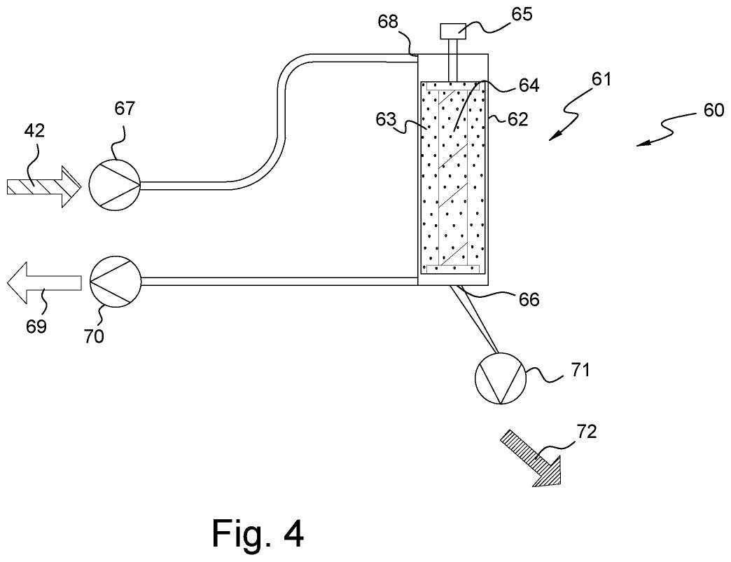

According to an embodiment thereof shown in FIG. 4, third or thickener/dewatering module 60 includes a solid separator 61, that includes a case 62 under pressure, wherein a fine filter 63 and a coaxially inner worm screw 64 are arranged. While worm screw 64 is driven to rotate slowly by motor 65 in a direction that tends to carry material downwards, the liquid part passes through filter 63, while the suspended solids are retained by filter 63 and pushed toward an outlet end 66 of solid separator 61. Screw 64 may rotate more slowly than worm screw 5. For example, screw 64 may be driven to rotate at a rate of about 50 rpm to about 400 rpm.

Third or thickener/dewatering module 60 may further include a loading pump 67 for inputting the slurry 42 (FIG. 1) output from settling module 30 into an inlet 68 of solid separator 61. It will be understood that either one of pumps 43 and 67 may be provided for.

The liquid part of input slurry 42, that passes through filter 63, forms a clarified effluent or diluted stream 69 (FIG. 1), which may simply drain off from third module 60; alternatively, third or thickener/dewatering module 60 may optionally include a drain pump 70 therefor.

Third or thickener/dewatering module 60 further includes a drain pump 71 for extracting the more concentrated stream, that includes the suspended solids of input slurry 42 that have been retained by filter 63, and that forms slurry 72 (FIG. 1) output from third module 60, referred to as dewatered slurry 72 herein. The speed of the drain pump 71 for the dewatered slurry 72 determines, in part, the dry matter content of dewatered slurry 72. When arranged directly at the output 66 of solid separator 61, drain pump 71 may also serve to make water-tight the solid separator 61, and to create a counter-pressure within solid separator 61, allowing the solids to be “squeezed” so as to increase the amount of water or liquid into the diluted stream 69 and to increase the concentration of dewatered slurry 72.

Pumps 67, 70, 71 are preferably suitable positive displacement pumps such as lobe pumps.

Turning back to FIG. 1, the dewatered slurry 72 output from third or thickener/dewatering module 60 typically has a dry matter content of about 10% to about 20% e.g. about 12-15%. As further discussed below, this allows reducing the volume of the downstream digester 90, the flowrate of treated wet fraction of packaged waste stream 3 being equal; or vice versa it allows, the volume of the downstream digester 90 being equal, to increase the flowrate of treated wet fraction of packaged waste stream 3 and the methane output from plant 1. Too low values of dry matter contents of dewatered slurry 72 would not justify the investment costs of providing thickener/dewatering module 60, though being easily achievable. The higher limit may be dictated by mechanical limitations.

Dewatered slurry 72 is thicker than slurry 42 input to thickener/dewatering module 60 and thicker than slurry 26 output from first or squeezing-diluting-unpacking module 4; accordingly, slurry 26 is referred to as being comparatively liquid, and slurry 72 is referred to as being comparatively dewatered herein and in the attached claims.

The diluted stream 69 has a relatively low dry matter content. For example, diluted stream 69 may have a dry matter content of 4% or less. In some embodiments diluted stream 69 may have a dry matter content of 3.5% or less or 3% or less. It is desirable to operate treatment plant 1 so that dry matter content of diluted stream 69 is low. In example cases the dry matter content of diluted stream 69 is in the range of about 2% to about 3.5%.

Diluted stream 69 is a heterogeneous aqueous solution. This liquid, essentially dirty water, is advantageously totally recirculated or recycled. Advantageously, the diluted stream 69 can be used to provide, at least in part, dilution fluid 10 for first or squeezing-diluting-unpacking module 4.

To this end, the diluted stream 69 output from the third or thickener/dewatering module 60 is collected in a tank, in short named water tank 73. Diluted stream 69 may form a major part (e.g. about 80-95%, preferably about 90%) of the dilution water stored in water tank 73, the remaining part (e.g. about 5% to 20%, for example about 10%) being fresh water or dirty water that is recirculated further downstream of the process, as will be described later on.

Third or thickener/dewatering module 60 may be designed with the dual aim of: (i) thickening the dewatered slurry 72 that will be fed, essentially unchanged, to the digester 90 in order to reduce its volume/increase the flowrate of stream of packaged waste 3, and (ii) obtaining the diluted stream 69 that will form, at least in part, dilution fluid 10 for first or squeezing-diluting-unpacking module 4.

Thus, thanks to the presence of downstream third or thickener/dewatering module 60 and to the water recycling, a large amount of water may be and is indeed input into first or squeezing-diluting-unpacking module 4. Thus, the slurry processed in the first two modules 4, 30 may be kept more diluted, e.g. at a ratio of waste wet fraction to water about 1:5 to 1:10. For example, the ratio of waste wet fraction to fluid 10 may be about 1:7 or 1:10 and in some embodiments is about 1:8. This ensures that, irrespective of the dry matter content of the stream of packaged waste 3, the slurry treated by the first two modules 4, 30 has a very low dry matter content, say <=6%.

This has the following advantages:
the organic matter is more efficiently rinsed off from the large inerts extracted by the first module 4,
the removal of fine inerts in second or settling module 30 is very efficient, because a very high and stable (constant) water content aids settling of inerts,
the dry matter contents and thus the slurry quality overall treated in plant 1 is very little affected by the specific waste wet fraction forming packaged waste stream 3 to the plant 1 at any time,
the flow rate of the dewatered slurry treated in the plant 1, notably in the downstream digester 90, is very constant, this helps to make operation of digester to yield gas more efficient and more consistent. Indeed, the digestion activity is carried out by bacteria, and the organic waste may be considered as food for the bacteria: the more stable is the food quality and quantity, the more stable is the bio-gestion process and biogas production.

For a better understanding of the third advantage mentioned above, it is noted that in prior art plants missing a thickener/dewatering module (cf. third module 60), the ratio waste wet fraction to water within an input diluting module (cf. first module 4), which ratio is also essentially kept in a subsequent settling module (cf. second module 30), is usually about 1:2. Since, as said, the waste wet fraction has a dry matter contents that is comparatively highly variable (e.g. 15% to 40%), the overall dry matter contents of the slurry produced by such prior art plants and fed to a digester is also highly variable depending on the specific waste wet fraction input to the plant at any time. This variability can result in inefficient digestion.

Irrespective of whether dilution fluid 10 is provided from recycled liquid stored in water tank 73 or not, dilution fluid 10 is advantageously metered to the first or squeezing-diluting-unpacking module 4 by a control system configured to automatically adjust the rate at which dilution fluid 10 is provided to first module 4 to achieve a desired dry matter content in the slurry downstream from first module 4. Such a control system may, for example, comprise a controller 75 connected to control an electronically actuated valve (e.g. an electronically adjustable proportional flow control valve operated by a solenoid or other electrical actuator) 74 in a feedback controlled manner.

Specifically, controller 75 provides the input signal of control valve 74, determining its percentage opening and thus the amount of dilution fluid 10 sprayed into first module 4, so as to obtain a desired process value or set-point for the dry matter content of the processed slurry upstream of the third or thickener/dewatering module 60, that represents the measured process value.

More precisely, a dry matter content sensor 76 is provided. Sensor 76 may, for example, be an optical sensor, a microwave sensor, or a radiation sensor or the like. As an alternative thereto, a humidity sensor may be used, and then the complement to 100% of its output may be computed to provide the measured process value.

From a logical point of view, sensor 76 should be placed between the first module 4 and the second module 30, as shown in FIG. 1. In a less preferred variant, sensor 76 is arranged, from a logical point of view, downstream of second module 30, as indicated in phantom by sensor 76a in FIG. 1. In a third variant, sensor 76 is arranged in an appropriate position within second module 30, as indicated in phantom by sensor 76b in FIG. 1, preferably before or after the inert removal process that occurs in module 30, in a containment chamber or along a main conduit or a branch conduit, also depending on the implementation of the sensor itself. Sensor 76b is suitably embodied by the above mentioned sensor 50, that is arranged within basin 31 below the slurry level, or by sensor 50a arranged in one of the pipes 49a, 49b associated with recirculation pump 49.

Controller 75 may be a simple controller that adjusts the amount of dilution liquid added at first module 4 based on a deviation from a target dry matter content at a location downstream from first module 4. In some implementations controller 75 implements a proportional-integral-derivative controller (P.I.D. controller), which performs control based on up to a three-fold analysis, that takes into account the current difference between the measured dry matter content and a set-point thereof (P), the past values of such difference (I), and how fast the difference is changing (D).

Specifically, controller 75 may implement the following general function::

$$u ( t ) - {K}_{p} ( e ( t ) + \frac{1}{{𝒯}_{i}} {\int }_{\prod }^{t} e ( {t}^{′} ) {dt}^{′} + {T}_{d} \frac{de ( t )}{dt} ) ,$$

wherein:
u(t) is a signal controlling opening of the control valve 74, expressed in terms of percentage;
e(t) is a signal representing the difference between the measured dry matter content
M(t) and a set-point thereof M0,
Kp is a proportionality constant which value is selectable to tune operation of the control;
Ti is the integration time, and
Td is the derivative time..

As mentioned above, it is advantageous for slurry 26 to have a dry matter content in the range of about 4% to 6%. The set point M0 may be a value in this range (e.g. 5% or 5.5% or 4.5% etc.).

Turning back to the dewatered slurry 72 output from third or thickener/dewatering module 60, is preferably stored in a buffer unit 77 from which dewatered slurry 72 is fed to digester 90 as slurry 78, though it will be understood that slurry 78 may be essentially the same as slurry 72.

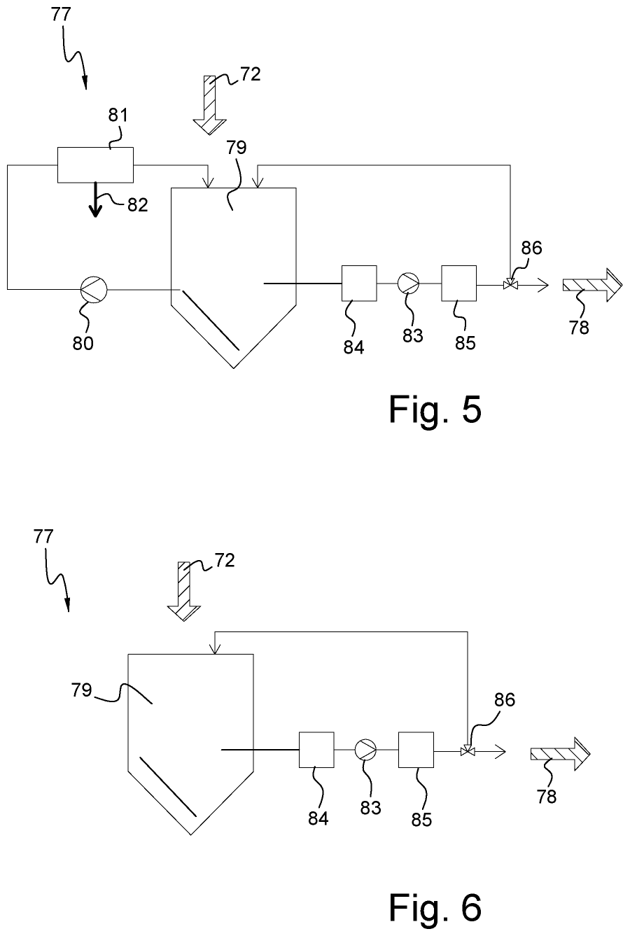

According to an embodiment thereof diagrammatically shown in FIG. 5, buffer unit 77 comprises, in a manner known per se, a tank 79 with a tapered bottom. A pump 80 feeds dewatered slurry 72 from the bottom of tank 79 through a sand trap 81, which may be similar to the second or settling module 30, back to tank 79, while sand and other inerts are discharged as shown by inert stream 82 (FIG. 1).

From a higher level within tank 79, the dewatered slurry 72 is pumped by pump 83 through at least one and preferably two grinders 84, 85, one upstream of and one downstream of pump 83, back to tank 79 or to the digester 90 as controlled by a three-port valve 86.

In greater detail, pump 83 may feed slurry 78 to downstream digester 90 at a constant hourly flow rate, according to a programmable cyclic loading prescription, with one-hour cycle; when not being pumped to digester 90, dewatered slurry 72 may be sent back to tank 79 of buffer unit 77; alternatively pump 83 may be simply switched off during non-loading periods. Pump 83 is diagrammatically represented as being external to buffer unit 77 in FIG. 1.

This embodiment of buffer unit 77 ensures that all the dewatered slurry 72 is continually ground in a recirculated manner while it is not fed as slurry 78 to the downstream digester 90, so that the slurry 78 that is eventually fed to the digester 90 is finely ground. This lowers the risk of clogging of the conduits to digester 90, and of settling in the conduits and in the digester 90. Because the organic matter is finely ground by grinders 84, 85, then within the downstream digester 90, there will be no comparatively large solid pieces suspended in the slurry, and the contact surface between organic matter and bacteria will be large. A large contact surface area helps to enhance digestion.

As shown in the preferred, simplified embodiment of buffer unit 77 of FIG. 6, the recirculation of the slurry through sand trap 81 through pump 80 can be dispensed with. This is because the slurry 72 has been dewatered by the use of third or thickener/dewatering module 60, and the high concentration of dry matter in the dewatered slurry 72 tends to keep any non-removed inerts suspended, possibly not allowing them to settle.

Turning back to FIG. 1, digester 90 may, for example, be a thermophilic (temperature 55° C.) or a mesophilic (38-39° C.) digester. Suitable digesters are commercially available and are well known.

The residence time for slurry 78 in digester 90 is a very important process parameter, representing the time needed for the bacteria to digest the material and degrade it to yield products which can include methane and water. The process efficiency is closely related to the residence time. A typical design value may be for example 15-40 days, preferably 30-35 days. The residence time equals the ratio of digester volume (e.g. expressed in m³) to volume flowrate (e.g. expressed in m³/day).

The amount of methane produced by digester 90, indicated by methane stream 91 in FIG. 1, is directly proportional to the amount of dry matter fed to digester 90, which is another important process parameter, expressed in weight of dry matter per unit time (e.g. tons DM/day).

The amount of dry matter fed to digester 90 per unit time (expressed e.g. in tons DM/day) depends on the concentration of dry matter (tons DM/m³) in slurry 83, which is essentially equal to that in dewatered slurry 72.

Thus, once the design amount of waste that should be treated by the plant 1 per unit time (expressed e.g. in tons/day of the material in packaged waste stream 3) has been set, and the residence time being equal, then third or thickener/dewatering module 60 has the effect of lowering the volume needed for the digester 90. Namely, the volume of the digester 90 may be reduced when it has to treat a dewatered slurry 72 (78) exiting from third or thickener/dewatering module 60 having a high dry matter content, compared to the case where slurry 42 output from the second or settling module 30 having a low dry matter content is directly fed to digester 90. This brings about advantages in terms of invested capital and of occupied surface, as well as lower operating costs for heating digester 90.

Conversely, if digester 90 has a set volume (e.g. in case of an upgrade of an existing plant), adding third or thickener/dewatering module 60 and operating the plant as described herein allows, the residence time being equal, a larger packaged waste stream 3 to be treated by plant 1, and thus a larger amount of methane 91 to be produced (of course, adapting the remaining components, notably the first and second modules 4, 30).

A metering pump 92 takes out metered amounts of digestate 93 so as to keep the slurry level within digester 90 essentially constant, and sends digestate 93 to a solid separator 94.

Solid separator 94, per se well known, can be a centrifuge that separates the solid digestate from the liquid part. The solid digestate stream 95 is sent to composting or to other treatment units, like drying, carbonation (treatment with lime and carbon dioxide) or to external disposal as waste.

The liquid stream 96 may essentially be dirty water with a dry matter content of about 0.05-2%, preferably of about 0.1% (about 1 g/l). Liquid stream 96 is sent to a per se well-known waste water treatment plant 97.

A part of the water collected in waste water treatment plant 97 is sent, as minor recycling stream 98, to water tank 73, and forms the above mentioned minor part (e.g. about 15-20%) of the dilution fluid 10 that is input to first module or squeezing-diluting-unpacking module 4, preferably under the control of controller 75 as discussed above.

It is unimportant whether this minor recycling stream 98 is actually purified within waste water treatment plant 97 or not, and advantageously it will not be treated. The flowrate of the liquid to be purified by waste water treatment plant 97 is therefore advantageously unrelated to the flowrate of the dilution liquid 10 used to dilute the organic waste in first module or squeezing-diluting-unpacking module 4.

The rest of the liquid or dirty water collected in waste water treatment plant 97 is purified thereby, and then it can be discharged to sewerage, to surface water body (river or lake) or on the ground, as discharge stream 99.

The flowrate of discharge stream 99 is proportional to the humidity content of packaged waste stream 3—disregarding any water used to periodically clean the plant and/or prepare chemicals for use in the plant. The flowrate of the liquid to be purified by waste water treatment plant 97 is therefore advantageously only related to the humidity content of stream of packaged waste 3.

In other terms, even if the slurry treated by the first and second modules 4, 30 is highly diluted with the above mentioned advantages, still waste water treatment plant 97 does not need to treat a large quantity of liquid. Overall, from the point of view of the process, the entire plant 1 can operate, at full operation, without requiring any fresh water (apart from that used to clean the plant and/or prepare chemicals). This has environmental and operating cost benefits.

First module or squeezing-diluting-unpacking module 4, second module or settling module 30, any additional settling module 30a, and third or thickener/dewatering module 60 are preferably all included in a single structure, forming one and the same machine or apparatus 2. The advantage is that of having a compact machine, lowering the space usage. Furthermore, because all these modules can be kept in the same room as where the waste arrives, the risk of odorous emissions is lowered.

FIG. 7 diagrammatically shows an embodiment of such an apparatus 2 according to an advantageous embodiment.

First module or squeezing-diluting-unpacking module 4 is arranged immediately on top of second module or settling module 30, with the advantages outlined above; an additional settling module 30a is arranged next to second module or settling module 30; and third or thickener/dewatering module 60 is arranged partly on top of the additional settling module 30a and partly on top of one or more buffer basins 100, that may advantageously embody buffer unit 77 and/or water tank 73 and/or a buffer for slurry 42 between second module or settling module and third or thickener/dewatering module, circulation pipes and pump(s) being provided for as needed. A frame or a case 101 encompassing all the above components is also shown.

Preferably the structure of such apparatus 2 is formed from plural frames assembled together, wherein each module 4, 30, 30a, 60 has such a size as to allow shipment within a standard shipping container. Alternatively, first module or squeezing-diluting-unpacking module 4 and second module or settling module 30 may fit within one standard shipping container.

Alternatively, squeezing-diluting-unpacking module 4, second module or settling module 30, any additional settling module 30a, and third or thickener/dewatering module 60 may all be separated (especially in case of retrofitting); or still alternatively, squeezing-diluting-unpacking module 4, and second module or settling module 30 (and any additional settling module 30a) can be in a same apparatus while third or thickener/dewatering module 60 may be separated therefrom.

Preferably, such apparatus 2 integrates sensor 76 (76a, 76b), control valve 74 and controller 75 (not shown in FIG. 7).

It will be understood that the apparatus described herein may be varied without departing from the inventive concepts described herein. One manner of variation is the way in which the various modules described herein are laid out. For example, while it is convenient for first module 4 and second module 30 to be co-located and combined it is possible in the alternative that modules 4 and 30 are located separately. Similarly, second module 30 may include any suitable number of settling vessels and these settling vessels may be connected in series and/or in parallel and may be located together or at spaced apart locations. Suitable conveyors, pumps or other delivery systems may be provided to deliver a stream from one module to another module. Buffer vessels may optionally be provided between modules.

In the description above, any motor that is shown directly connected to a driven component may instead be indirectly connected to the component, through any suitable transmission mechanism such as a belt transmission, a gear train, a hydraulic drive, and similar. Furthermore, one single motor may replace two or more motors and used to drive two or more components, through any suitable transmission mechanism.

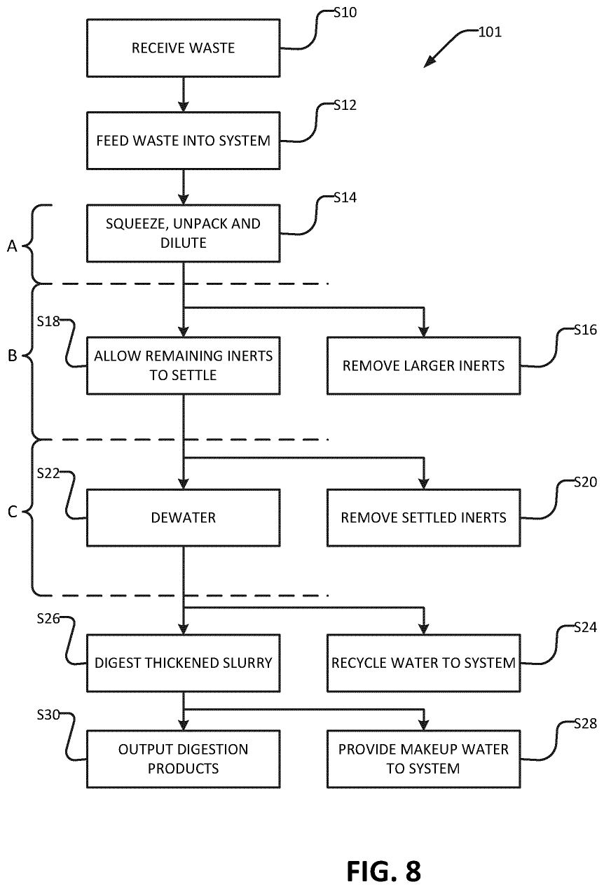

FIG. 8 is a flow chart illustrating a method 101 according to an example embodiment. At S10 organic waste is received. The Organic waste has the characteristics of OFMSW and/or industrial organic waste (e.g. organic materials such as waste food and/or food precursors mixed with significant quantities of packaging and/or other inerts.

The dry matter content of the organic waste received at S10 may vary significantly over time as a result of the organic waste arising from different sources and/or changing in composition from the same sources.

At S12 the organic waste is fed into a system for preprocessing (for example a system as described herein. At S14 the organic waste is squeezed, unpacked and diluted. The dilution performed at S14 may comprise controlled addition of water such that a resulting slurry has a dry matter content at or near a set point notwithstanding the variation in dry matter content of organic waste provided by S12. The dilution provides a slurry having a dry matter content significantly lower than that of the incoming organic waste.

For example, diluting the packaged waste stream may comprise adding water to the packaged waste stream in a volume ratio in the range of one part packaged waste stream to five parts water to one part packaged waste stream to ten parts water (e.g. a volume ratio of about one part packaged waste stream to about eight parts water).

The unpacking performed at S14 may comprise mechanically (e.g. through the action of blades, water jets or the like) tearing, shredding, crushing or grinding packaging and/or other inerts. This facilitates separation of the organic waste from the inerts and facilitates separation of the inerts from a stream (e.g. a slurry as described above) containing the organic waste.

At S16 larger inerts are removed (e.g. by use of a filter comprising a screen, mesh, perforated plate or the like that is arranged to segregate the larger inerts from a fraction containing the organic waste (e.g. the slurry discussed above).

At S18 remaining inerts are allowed to settle so that they can be collected.

At S20 the settled inerts are removed.

At S22 the organic waste (less the settled inerts) is dewatered. This thickens the organic waste in preparation for digestion.

At S24 water removed at S22 is recycled (e.g. to S14).

At S26 the dewatered slurry is digested. S26 may include extracting bio gas from the digesting slurry and/or generating electricity from the digesting slurry.

At S28 water from the digestion process is fed back to an earlier step as makeup water. The rate at which makeup water is provided by S28 may be much smaller (e.g. less than 15% or less than 10%) than the rate at which recycled water is provided by S24.

At S30 the digested slurry is output.

In method 101, the dry matter content of a stream containing the organic materials being processed varies as the method progresses. In region A the dry matter content is variable but typically in the range of about 15% to about 40% and is generally in the range of about 25-30%. In region B the dry matter content may be controlled to be in the range of about 4% to about 6% e.g. about 3.5% to about 6%. In region C the dry matter content is typically in the range of about 10% to about 20% e.g. about 12-15%.

This description of the exemplary embodiments is intended to be read in connection with the accompanying drawings, which are to be considered part of the entire written description. In the present description and in the following claims, all of the numerical magnitudes indicating quantities, parameters, percentages, and so on should in all circumstances be deemed to be preceded by term “about”, unless otherwise indicated. Furthermore, all of the ranges of numerical magnitudes include all of the possible combinations of maximum and minimum numerical values and all of the possible intermediate ranges, besides those specifically indicated hereinafter. In the description, relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

As used herein, the term “about” means that a stated numerical value or values is approximate and small variations would not significantly affect the practice of the disclosed embodiments. Where a numerical limitation is used, unless indicated otherwise by the context, “about” means the numerical value can vary by ±10% and remain within the scope of the disclosed embodiments. For example “about 10” can mean the range from 9 to 11.

The above is a description of various embodiments of inventive aspects, and further changes can be made without departing from the scope of the present invention. The shape and/or size and/or location and/or orientation of the various components and/or the succession of the various steps can be changed. The functions of an element or module can be carried out by two or more components or modules, and vice-versa. Components shown directly connected to or contacting each other can have intermediate structures arranged in between them. Steps shown directly following each other can have intermediate steps carried out between them. The details shown in a figure and/or described with reference to a figure or to an embodiment can apply in other figures or embodiments. Not all of the details shown in a figure or described in a same context must necessarily be present in a same embodiment. Features or aspects that turn out to be innovative with respect to the prior art, alone or in combination with other features, should be deemed to be described per se, irrespective of what is explicitly described as innovative.

1. An apparatus (2) for pretreatment of organic waste from the Organic Fraction of Municipal Solid Waste and/or from food industries, upstream of an anaerobic digester, the apparatus comprising:
a squeezing-diluting-unpacking module (4) configured to receive an input waste stream (3) of packaged waste and a dilution fluid (10), to lacerate packaging of the packaged waste, and to output a coarser non-digestible stream (23) and a comparatively dirty and comparatively liquid slurry (26),
at least one settling module (30, 30a) connected to receive the comparatively dirty and comparatively liquid slurry (26) and configured to allow inerts in the comparatively dirty and comparatively liquid slurry (26) to sediment and to output a finer inerts stream (40) and a comparatively refined slurry (42),
a thickening/dewatering module (60) connected to receive the comparatively refined slurry (42) and configured to separate and output a liquid stream (69) and a comparatively dewatered slurry (72) wherein the comparatively dewatered slurry (72) is output to the anaerobic digester and the liquid stream (69) is connected to supply liquid making up at least part of the dilution fluid (10) received at squeezing-diluting-unpacking module (4).

2. The apparatus (2) of claim 1, further comprising:
a dry matter content sensor (50, 50a, 76, 76a, 76b) arranged between said squeezing-diluting-unpacking module (4) and said at least one settling module (30, 30a), within said at least one settling module (30, 30a), or downstream of said at least one settling module (30, 30a);
a control valve (74) configured to meter the dilution fluid (10) input to squeezing-diluting-unpacking module (4); and
a controller (75) configured to receive an output signal from the dry-matter content sensor (50, 50a, 76, 76a, 76b) and to provide a control signal input to control valve (74) so as to control the dry matter content of the comparatively dirty and comparatively liquid slurry (26) or of the comparatively refined slurry (42), according to where the sensor (50, 50a, 76, 76a, 76b) is arranged, to match a set-point.

3. The apparatus (2) of claim 2, wherein said controller (75) is a P.I.D. controller.

4. The apparatus (2) of claim 1, wherein the comparatively dirty and comparatively liquid slurry (26) has a dry matter content in the range of about 4-6%, and the comparatively dewatered slurry (72) has a dry matter content in the range of about 10-20%.

5. The apparatus (2) of claim 1, wherein the settling module (30) includes an elongate basin (31), the basin including at least one chamber (32, 32a), a worm screw (34, 34a) extending on the bottom thereof and a motor operable to drive rotation of the worm screw (34) at a comparatively low speed for collecting the inerts that sediment from the comparatively dirty slurry (26).

6. The apparatus (2) of claim 5, wherein the bottom portion of the elongate basin (31) of the settling module (30) is divided into at least two chambers (32, 32a), each chamber having tapered walls (33, 33a), and the worm screw (34, 34a) is a first worm screw extending on the bottom of one respective chamber (32, 32a) and one or more other worm screws (34. 34a) respectively extend on the bottom of the other respective ones of the chambers (32, 32a).

7. The apparatus (2) of claim 1, wherein the squeezing-diluting-unpacking module (4) includes an upwardly conveying worm screw (5) rotating at a comparatively high speed within a cylindrical filter (6) enclosed by a nozzled manifold (9) for inletting the dilution fluid (10).

8. The apparatus (2) of claim 1, wherein the thickening/dewatering module (60) includes a solid separator (61) that includes a case (62) under pressure, a fine filter (63) and a coaxially inner worm screw (64) being arranged in case (62), the inner worm screw (64) rotating at a comparatively low speed.

9. The apparatus (2) of claim 1, wherein said squeezing-diluting-unpacking module (4), said at least one settling module (30, 30a), and said thickener/dewatering module (60) are all included in a single structure, forming one and the same machine.

10. The apparatus (2) of claim 9, wherein the structure of said machine is formed from plural frames assembled together, wherein each of said squeezing-diluting-unpacking module (4), said at least one settling module (30, 30a), and said thickener/dewatering module (60) has such a size as to allow shipment with a standard shipping container, or wherein the squeezing-diluting-unpacking module (4) and the settling module (30) together have such a size as to allow shipment with a standard shipping container.

11. A plant (1) for pretreatment of organic waste from the Organic Fraction of Municipal Solid Waste and/or from food industries comprising an apparatus (2) according to claim 1, and the anaerobic digester (90) downstream thereof.

12. The plant (1) of claim 11, further comprising a solid separator (94) downstream of the anaerobic digester (90), and a waste water treatment plant (97) downstream of the solid separator (94), wherein a remaining part of the dilution fluid (10) input to squeezing-diluting-unpacking module (4) is provided by liquid collected, but not treated, by the waste water treatment plant (97).

13. The plant (1) of claim 11, further comprising a buffer unit (77) arranged between the thickening/dewatering module (60) and the anaerobic digester (90), the buffer unit (77) comprising a tank (79) storing the comparatively dewatered slurry (72), and a pump (83) selectively pumping the comparatively dewatered slurry (72) through at least one grinder (84, 85) to the downstream anaerobic digester (90).

14. The plant (1) of claim 13 wherein the buffer unit (77) lacks any recirculation of the comparatively dewatered slurry (72) through a sand trap.

15. A method for pretreatment of organic waste from the Organic Fraction of Municipal Solid Waste and/or from food industries, upstream of an anaerobic digester, the method comprising:
a) receiving a packaged waste stream,
b) squeezing and diluting the packaged waste stream while unpacking the waste to output a coarser non-digestible stream and a comparatively dirty and comparatively liquid slurry,
c) letting comparatively finer inerts in the comparatively liquid slurry sediment to output a finer inerts stream and a comparatively refined slurry,
d) thickening/dewatering the comparatively refined slurry to output a liquid stream and a comparatively dewatered slurry;
e) delivering the comparatively dewatered slurry to the anaerobic.digester and delivering liquid from the liquid stream for diluting the packaged waste stream in the step b).

16. The method of claim 15, further comprising:
measuring a dry matter content of the comparatively dirty and comparatively liquid slurry (26) or of the comparatively refined slurry (42); and
metering a dilution fluid of said step b) to control the measured dry matter content to match a set-point, SP.

17. The method of claim 16 wherein the set-point, SP, corresponds to a dry matter content of less than 6% and the method comprises maintaining the dry matter content of the comparatively dirty and comparatively liquid slurry to be in the range of SP±1%.

18. The method of claim 16 comprising maintaining the dry matter content of the comparatively dirty and comparatively liquid slurry to be in the range of 4% to 6%.

19. The method of claim 15 comprising using the liquid stream of the step d) for diluting the packaged waste stream in the step a).

20. The method of claim 20 comprising digesting the comparatively dewatered slurry, and supplying water output by the digesting for diluting the packaged waste stream in the step b).

21. The method of claim 15 wherein diluting the packaged waste stream in the step b) comprises adding water to the packaged waste stream in a volume ratio in the range of one part packaged waste stream to five parts water to one part packaged waste stream to ten parts water.

22. The method of claim 21 wherein diluting the packaged waste stream in the step b) comprises adding water to the packaged waste stream in a volume ratio of about one part packaged waste stream to about eight parts water.

23. The method of claim 15 wherein the coarser non-digestible stream comprises plastic from plastic bags.
